(19) United States
(12) Patent Application Publication
(10) Pub. No.: US 2026/0283630 A1
(43) Pub. Date: Sep. 24, 2026

(54) SYSTEMS, DEVICES, AND METHODS FOR TREATING VESSEL OCCLUSIONS

(71) Applicant: Covidien LP, Mansfield, MA (US)

(72) Inventors: Danyong ZENG, Aliso Viejo, CA (US); Guigui WANG, Aliso Viejo, CA (US); Tejashri KUMAR, Fountain Valley, CA (US); Ajay S. KADRENAHALLY, Rancho Santa Margarita, CA (US)

(21) Appl. No.: 19/533,431

(22) Filed: Feb. 9, 2026

Related U.S. Application Data
(60) Provisional application No. 63/756,922, filed on Feb. 11, 2025.

(51) Int. Cl. A61B 17/22; A61B 17/00

(52) U.S. Cl. CPC A61B 17/22; A61B 2017/00292; A61B 2017/00367; A61B 2017/00867; A61B 2017/22079

(57) ABSTRACT

Treatment devices and associated methods and systems are disclosed herein. According to some embodiments, an aspiration system includes a catheter having a proximal end, a distal end, and a lumen extending therebetween. The system further includes a vacuum source configured to provide vacuum to the lumen and an actuation mechanism coupled to the distal end of the catheter and configured to selectively extend the distal end in a distal direction by a controlled distance, such that extension of the distal end facilitates engagement and retrieval of a clot.

### CROSS-REFERENCE TO RELATED APPLICATIONS

This application claims the benefit of U.S. Provisional Patent Application Serial No. 63/756,922, filed February 11, 2025, the entire content of which is incorporated herein by reference.

### TECHNICAL FIELD

The present technology relates to systems and methods for removing obstructions from body lumens. Some embodiments of the present technology relate to aspiration catheters and associated components.

### BACKGROUND

Many medical procedures use medical device(s) to remove an obstruction (such as clotting material) from a body lumen, vessel, or other organ. An inherent risk in such procedures is that mobilizing or otherwise disturbing the obstruction can potentially create further harm if the obstruction or a fragment thereof dislodges from the retrieval device. If all or a portion of the obstruction breaks free from the device and flows downstream, it is highly likely that the free material will become trapped in smaller and more tortuous anatomy. In many cases, the physician will no longer be able to use the same retrieval device to again remove the obstruction because the device may be too large and/or immobile to move the device to the site of the new obstruction.

Procedures for treating ischemic stroke by restoring flow within the cerebral vasculature are subject to the above concerns. The brain relies on its arteries and veins to supply oxygenated blood from the heart and lungs and to remove carbon dioxide and cellular waste from brain tissue. Blockages that interfere with this blood supply eventually cause the brain tissue to stop functioning. If the disruption in blood occurs for a sufficient amount of time, the continued lack of nutrients and oxygen causes irreversible cell death. Accordingly, it is desirable to provide immediate medical treatment of an ischemic stroke.

To access the cerebral vasculature, a physician typically advances a catheter from a remote part of the body (typically a leg) through the abdominal vasculature and into the cerebral region of the vasculature. Once within the cerebral vasculature, the physician deploys a device for retrieval of the obstruction causing the blockage, for example an aspiration catheter. Concerns about dislodged obstructions or the migration of dislodged fragments increases the duration of the procedure at a time when restoration of blood flow is paramount. Furthermore, a physician might be unaware of one or more fragments that dislodge from the initial obstruction and cause blockage of smaller more distal vessels. Accordingly, there remains a need for improved devices and methods that can remove occlusions from body lumens and/or vessels.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a side view of an example treatment system in accordance with examples of the present technology.

FIG. 2A is a side view of a distal end portion of a treatment device in accordance with examples of the present technology.

FIG. 2B is a side view of the distal portion of the treatment device of FIG. 2A in an extended configuration.

FIG. 3 is a chart of vacuum pressure and actuation over time according to one example.

FIG. 4A is a cross-sectional view of a single-lumen implementation of the distal end portion of the treatment device taken along line 4-4 in FIG. 2B.

FIG. 4B is a cross-sectional view of dual-lumen implementation of the distal end portion of the treatment device taken along line 4-4 in FIG. 2B.

FIG. 5A is a side cross-sectional view of a treatment system with a tension-wire actuation element in an unextended configuration in accordance with examples of the present technology.

FIG. 5B is a side cross-sectional view of the treatment system of FIG. 5B with the tension-wire actuation element in an extended configuration.

FIG. 6A is a side cross-sectional view of a treatment system with a shape memory actuation element in an unextended configuration in accordance with examples of the present technology.

FIG. 6B is a side cross-sectional view of the treatment system of FIG. 6A with the shape memory actuation element in an extended configuration.

### DETAILED DESCRIPTION

The present disclosure relates generally to aspiration catheters for removing clots from blood vessels, and more particularly to aspiration catheters having an extendable distal end that can be selectively actuated during a clot removal procedure. Traditional aspiration catheters often face challenges in maintaining effective engagement with clots due to vessel anatomy and variations in clot composition, which can lead to incomplete clot removal or fragmentation. These challenges are particularly evident when attempting to achieve successful first-pass clot retrieval in the tortuous anatomy of the neurovasculature.

The aspiration catheters described herein address these challenges through a distal extension mechanism that allows the distal end of the catheter to be selectively extended while engaging a clot. In various embodiments, the extension mechanism may include a tension wire system, a shape memory element activated by electrical current, or combinations thereof. This extension capability enables better engagement with the clot by allowing the catheter tip to be extended distally after initial contact is made with the proximal end of the clot. The extension mechanism can be synchronized with the application of vacuum, such that the distal end extends during vacuum off periods to progressively engage more of the clot during the retrieval procedure.

In some embodiments, a pulsed vacuum source is used in conjunction with the distal extension mechanism to create a coordinated clot engagement and retrieval process. The catheter may be provided in either a single-lumen or dual-lumen configuration, with the dual-lumen embodiments including an outer lumen that can be extended relative to an inner aspiration lumen. The selective extension of the distal end, which may be in the range of about 10 to 50 millimeters in some examples, helps ensure comprehensive clot engagement while minimizing the risk of clot fragmentation or loss during the retrieval procedure. This approach represents a significant advancement over conventional aspiration catheter designs by providing active control over the catheter-clot interface throughout the retrieval process.

Various other features and aspects of example treatment systems and devices are described in more detail below. The present technology provides systems, devices, and methods for treating various medical conditions, including removing a thrombus or other occlusion from a bodily lumen. Although many of the embodiments are described below with respect to devices, systems, and methods for treating a cerebral or intracranial embolism, other applications and other embodiments in addition to those described herein are within the scope of the technology. For example, the treatment systems and methods of the present technology may be used to remove emboli from body lumens other than blood vessels (e.g., the digestive tract, etc.) and/or may be used to remove emboli from blood vessels outside of the brain (e.g., pulmonary, abdominal, cervical, or thoracic blood vessels, or peripheral blood vessels including those within the legs or arms, etc.). In addition, the aspiration systems and methods of the present technology may be used to remove luminal obstructions other than clotting material (e.g., plaque, resected tissue, foreign material, etc.). Additionally or alternatively, the treatment systems and devices described herein can be used for fluid delivery to a treatment site within a bodily lumen (e.g., delivery of medicament, saline, contrast media, or other suitable fluid to an intravascular treatment site within a cerebral vessel or other desired treatment site).

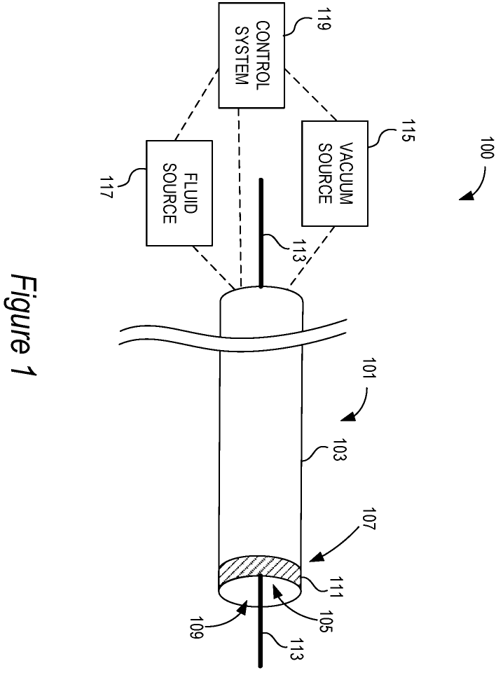

FIG. 1 illustrates a side view of a treatment system 100 in accordance with examples of the present technology. The treatment system 100 includes a treatment device 101, which in some implementations can be configured as an aspiration catheter. The treatment device 101 comprises a tubular body 103 having a lumen 105 extending therethrough. In some implementations, the tubular body 103 can be constructed using one or more polymeric materials suitable for intravascular use. These materials can include thermoplastic elastomers such as Pebax® (polyether block amides), polyurethanes, polyethylenes, polyamides (nylon), polysiloxanes, or combinations thereof arranged in a multi-layer configuration. The tubular body 103 can include reinforcement structures embedded within or between the polymer layers, such as metallic braids, coils, stents or hypotubes formed from stainless steel, nitinol, cobalt-chromium alloys, or other biocompatible metals. In some implementations, the reinforcement can include a braided structure with a pick count ranging from about 30 picks per inch (PPI) to about 300 PPI, with the pick count optionally varying along the length of the tubular body 103 to modify flexibility characteristics. The tubular body 103 include an inner liner formed from fluoropolymers such as polytetrafluoroethylene (PTFE) or fluorinated ethylene propylene (FEP) to provide a lubricious inner surface and can optionally include hydrophilic or hydrophobic coatings on exterior surfaces to enhance trackability through vasculature. Optionally, radio pacifiers such as Barium sulfate, Bismuth compounds and Tungsten material can be blended into polymer jacket materials (or other components of the tubular body 103) to increase radio-opacity.

For general intravascular applications, the tubular body 103 can have an outer diameter ranging from about 3 French (Fr) to about 24 Fr, and an inner diameter ranging from about 0.020 inches to about 0.200 inches, depending on the target vessel size and intended use. In implementations configured specifically for neurovascular applications, the tubular body 103 can have a smaller profile with an outer diameter ranging from about 3 Fr to about 8 Fr at its distal end, corresponding to approximately 1.0mm to 2.7mm, and an inner diameter ranging from about 0.020 inches to about 0.088 inches to enable navigation through the tortuous vessels of the neurovasculature while maintaining sufficient lumen size for clot aspiration. Additionally, or alternatively, the tubular body 103 can have a tapered configuration with a larger diameter at its proximal end transitioning to a smaller diameter at its distal end to balance trackability with aspiration efficiency.

The treatment device 101 includes a distal end portion 107 that, in some implementations, can be selectively actuated during a medical procedure. An actuation element 111 can be coupled to or integrated with the distal end portion 107. As described in more detail below, in some implementations, the actuation element 111 can be configured as a tension-wire assembly comprising one or more wires extending along the length of the catheter and coupled to the distal end portion 107. The tension-wire assembly can be configured such that applying tension to the wire maintains the distal end portion 107 in an unextended configuration, while reducing or releasing tension allows the distal end portion 107 to extend distally. Additionally or alternatively, the actuation element can be configured as a shape-memory assembly comprising a shape-memory material such as nitinol disposed at or near the distal end portion 107. The shape-memory assembly can be configured to transition from an unextended configuration to an extended configuration when heated above a transition temperature, such as by application of electrical current through conductors extending along the catheter body. In some implementations, the transition temperature can be selected to be above normal body temperature but below a temperature that could cause tissue damage.

The distal end portion 107 includes a distal opening 109 in fluid communication with the lumen 105. In operation, the distal opening 109 can be configured to engage and aspirate material such as clots or other occlusive material from a body lumen. As described in more detail below, manipulating the distal end portion 107 (e.g., extending the distal end portion 107 distally via the actuation element 111) can increase engagement of a clot with the distal opening 109 to increase first-pass efficacy.

The treatment system 100 can include a guide wire 113 that extends through the lumen 105. In some implementations, the guide wire 113 can be used to navigate the treatment device 101 through tortuous vasculature to reach a target treatment site. The guide wire 113 can be removably disposed within the lumen 105 such that it can be withdrawn or advanced relative to the treatment device 101 during a procedure.

A vacuum source 115 can be operably coupled to the treatment device 101. In some implementations, the vacuum source 115 can be configured to generate negative pressure within the lumen 105 to facilitate aspiration through the distal opening 109. The vacuum source 115 can be adjustable to provide various levels of negative pressure suitable for different procedural requirements. In various implementations, the vacuum source 115 can take the form of a pump, syringe, or other suitable device configured to supply negative pressure to the lumen 105.

The treatment system 100 can further include a fluid source 117 operably coupled to the treatment device 101. In some implementations, the fluid source 117 can be configured to deliver fluids through the lumen 105. The delivered fluids can include, for example, saline, contrast media, therapeutic agents, or combinations thereof. In various implementations, the fluid source 117 can take the form of a pump, syringe, etc. coupled to or including a reservoir containing fluid to be delivered through the lumen 105.

A control system 119 can be operably coupled to one or more components of the treatment system 100. In some implementations, the control system 119 can be coupled to the vacuum source 115 to control timing and amplitude of vacuum application. The control system 119 can also be coupled to the fluid source 117 to regulate fluid delivery through the lumen 105. Additionally, or alternatively, the control system 119 can be operably coupled to the actuation element 111 to control movements of the distal end portion 107. In some implementations, the control system 119 can coordinate the timing of vacuum application with actuation of the distal end portion 107 to optimize engagement with target material within a body lumen.

The control system 119 can include processing circuits configured to execute predetermined sequences of operations. In some implementations, the control system 119 can receive user inputs to modify operational parameters of the treatment system 100. The control system 119 can also include memory components storing operational sequences, threshold values, or other parameters related to operation of the treatment system 100. Communication between the control system 119 and other components can be achieved through various mechanisms. In some implementations, electrical connections, represented by dashed lines in FIG. 1, can couple the control system 119 to the vacuum source 115, fluid source 117, and actuation element 111. These connections can enable bidirectional communication for monitoring and control of the respective components. In various implementations, the control system 119 can include a tensioner and/or an electrical signal generator, as described in more detail below.

The components of the treatment system 100 can be arranged in various configurations while maintaining similar functionality. In some implementations, the vacuum source 115 and fluid source 117 can be integrated into a single unit. Additionally, or alternatively, the control system 119 can be integrated with either the vacuum source 115 or fluid source 117, or both, to provide a more compact system arrangement.

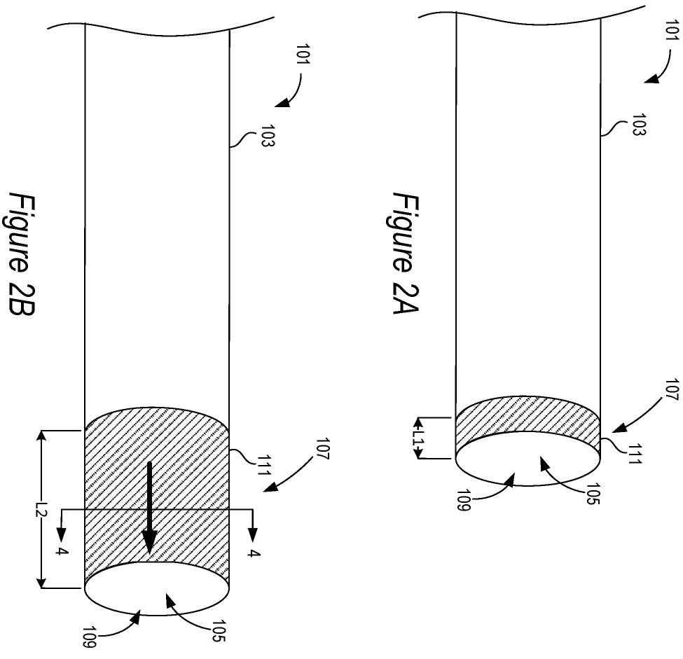

FIGS. 2A and 2B illustrate side views of a distal end portion of the treatment device 101 in an unextended configuration and an extended configuration, respectively, in accordance with examples of the present technology. As shown in FIG. 2A, the distal end portion 107 has an initial length L1 when in the unextended configuration. The actuation element 111 is coupled to or integrated with the distal end portion 107, and the distal opening 109 is configured to provide fluid communication with the lumen 105.

In FIG. 2B, the distal end portion 107 is shown in an extended configuration having a length L2, wherein L2 is greater than L1. In some implementations, the actuation element 111 can be operated to transition the distal end portion 107 from the unextended configuration (FIG. 2A) to the extended configuration (FIG. 2B). The extension from length L1 to length L2 can be performed in a single adjustment or, additionally or alternatively, can be performed incrementally through multiple intermediate positions between L1 and L2. In some implementations, the difference between L2 and L1 can range from about 10 millimeters to about 50 millimeters, for instance at least about 5, 10, 15, 20, 25, 30, 35, 40, 45, or 50 millimeters.

The actuation element 111 can be configured to maintain the distal end portion 107 at any selected position between and including the unextended configuration and the extended configuration. Additionally or alternatively, the actuation element 111 can be operated to reverse the extension, transitioning the distal end portion 107 from the extended configuration back toward the unextended configuration. In some implementations, this reversible extension capability enables progressive engagement with target material during a procedure while maintaining precise control over the position of the distal end portion 107.

In some implementations, the lumen 105 maintains a substantially consistent inner diameter during extension of the distal end portion 107, which can help ensure consistent aspiration performance throughout the range of motion. In some examples, the lumen 105 can be configured to expand and/or stretch radially outwardly to achieve a larger inner diameter during or instead of extension of the distal end portion 107, which may help engagement with larger clots. IIn some examples, the lumen 105 can be configured to compress radially inward, achieving a tapered shape or smaller diameter. This adjustment can facilitate catheter navigation to more distal anatomy. In some implementations, the distal end portion 107 can include a flexible outer jacket or covering that accommodates the extension while maintaining structural integrity of the device. The materials and construction of the distal end portion 107 can be selected to enable repeated extension and retraction cycles while resisting fatigue or degradation. In some implementations, the outer jacket material can be highly elastic, allowing it to extend under added force and shrink back to its original shape when the force is relieved.

FIG. 3 illustrates a timing diagram showing the relationship between vacuum pressure application and actuation events in accordance with examples of the present technology. The horizontal axis represents time, with time periods labeled T1 through T7, while the vertical axis represents pressure levels within the system. In some implementations, the vacuum pressure profile follows a square wave pattern, alternating between vacuum-off periods (shown at the baseline) and vacuum-on periods (shown at elevated levels).

The actuation events, represented by hatched vertical bars in FIG. 3, can be timed to occur during vacuum-off periods. In some implementations, each actuation event represents an increment of distal end extension. The timing diagram shows three separate actuation events spaced apart by predetermined intervals, with each actuation occurring during a distinct vacuum-off period. The duration of each actuation event can be shorter than the corresponding vacuum-off period to ensure completion of the actuation before vacuum is reapplied.

In some implementations, the vacuum-on periods (shown as elevated rectangular pulses) can have substantially consistent duration and amplitude. The vacuum-off periods between pulses can provide intervals during which the distal end portion can be extended without interference from aspiration forces. Additionally or alternatively, the timing between actuation events can be adjusted based on procedural requirements, such as the size or composition of target material being removed.

The control system can coordinate the timing of vacuum pulses and actuation events to optimize engagement with target material. In some implementations, the duration of time periods T1 through T7 can be adjusted to accommodate different procedural needs. Additionally or alternatively, the number of actuation events and their spacing can be modified based on real-time feedback during a procedure. The square wave pattern of vacuum application can help maintain consistent aspiration performance while allowing for controlled extension of the distal end portion during vacuum-off periods. As one example, each period T1-T7 may be about 10 seconds in length, though other durations are possible, and in some implementations the lengths of the vacuum-on and vacuum-off periods can differ.

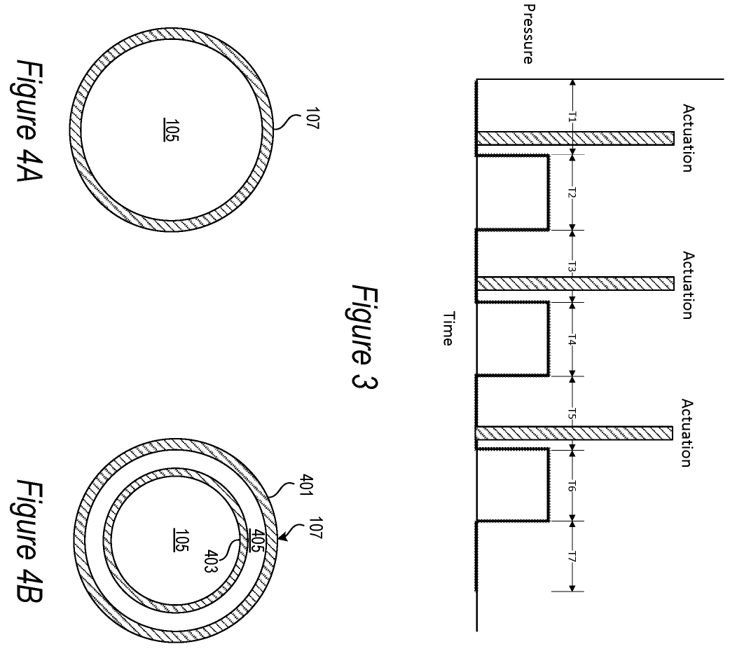

FIGS. 4A and 4B illustrate cross-sectional views of different implementations of the distal end portion 107, taken along line 4-4 in FIG. 2B. FIG. 4A shows a single-lumen implementation wherein the distal end portion 107 defines the lumen 105 extending therethrough. In some implementations, the wall of the distal end portion 107 can be formed from materials selected to accommodate extension and retraction while maintaining structural integrity and lumen patency.

FIG. 4B shows a dual-lumen implementation of the distal end portion 107, which includes an outer tubular element 401 and an inner tubular element 403 disposed within the outer tubular element 401. The inner tubular element 403 defines the lumen 105 extending therethrough, which in some implementations can serve as an aspiration lumen. An outer lumen 405 is defined in the annular space between the outer tubular element 401 and the inner tubular element 403. In some implementations, the outer tubular element 401 can be configured to move relative to the inner tubular element 403 during extension and retraction of the distal end portion 107. Additionally, or alternatively, the outer lumen 405 can be used for various purposes, such as fluid delivery, additional aspiration pathways, or housing components of the actuation element.

The components of either implementation can be formed from biocompatible materials suitable for intravascular use, for instance flexible polymers. In some implementations, the wall thickness of the tubular elements can be selected to provide appropriate flexibility while maintaining sufficient column strength for navigation through vasculature. The dimensions of the lumen 105 can be substantially consistent between the single-lumen and dual-lumen implementations to maintain similar aspiration performance characteristics. Additionally, or alternatively, the outer diameter of the distal end portion 107 can be substantially similar between implementations to ensure compatibility with similar vessel sizes.

In the dual-lumen implementation, the relative diameters of the inner tubular element 403 and outer tubular element 401 can be selected to provide appropriate clearance in the outer lumen 405 while maintaining a compact overall profile. In some implementations, the surfaces of the tubular elements can include coatings or surface treatments to reduce friction during relative movement. Additionally, or alternatively, sealing elements can be provided between the tubular elements to prevent fluid communication between the lumen 105 and the outer lumen 405.

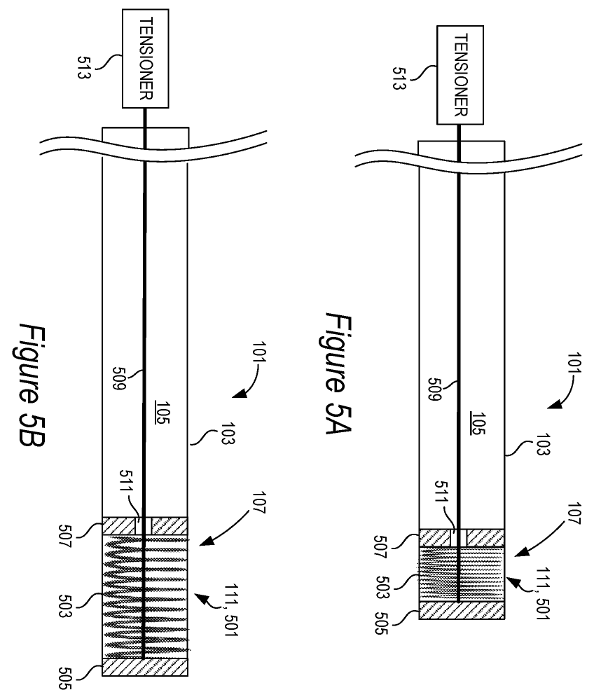

FIGS. 5A and 5B illustrate side cross-sectional views of an implementation of the treatment device 101 having an actuation element 111 configured as a tension-wire assembly 501. FIG. 5A shows the distal end portion 107 in an unextended configuration, while FIG. 5B shows the distal end portion 107 in an extended configuration. The tension-wire assembly 501 is configured to provide controlled extension of the distal end portion 107 while maintaining lumen patency throughout the range of motion.

The tension-wire assembly 501 includes a resilient member 503 disposed between a distal anchor 505 and a proximal anchor 507. In some implementations, the resilient member 503 can be configured as a coil spring formed from a biocompatible metal such as stainless steel, cobalt-chromium alloy, or nitinol. Additionally, or alternatively, the resilient member 503 can be formed from a superelastic material that provides consistent spring characteristics over multiple compression-extension cycles. The coil spring can be configured with various parameters, such as wire diameter, coil diameter, pitch, and number of turns, selected to achieve desired extension forces and distances. In some implementations, the spring constants can range from about 0.1 N/mm to about 10 N/mm. The resilient member 503 can include or be covered with a biocompatible super-stretchable polymer material, a jacket, sheath, biocompatible coating, etc.

The distal anchor 505 and proximal anchor 507 can be configured as tubular members that help maintain the patency of the lumen 105 during extension and compression of the resilient member 503. These anchor members can be formed from rigid or semi-rigid biocompatible materials such as metallic alloys, engineering polymers, or combinations thereof. Additionally or alternatively, the resilient member 503 can be constructed with a tubular configuration, such as a mesh-like structure or a series of interconnected rings, to help maintain lumen patency while providing the desired resilient characteristics. In various implementations the distal anchor 505 and/or the proximal anchor 507 can be made of radiopaque materials or may have radiopaque markers coupled thereto, thereby facilitating visualization of their respective positions under fluoroscopy.

A pull wire 509 extends through the lumen 105 and is coupled to the distal anchor 505. The pull wire 509 can be formed from materials selected to provide appropriate tensile strength and flexibility, such as stainless steel, nitinol, or high-strength polymeric fibers. In some implementations, multiple pull wires 509 can be used to provide balanced tensioning forces, with the pull wires optionally being spaced circumferentially around the lumen. The pull wire 509 passes through an aperture 511 in the proximal anchor 507 and extends proximally through the tubular body 103 to a tensioner 513 disposed at or near the proximal end of the treatment device 101. Among examples, the pull wire 509 can be routed inside lumen or it can attach to outside lumen, with a distal end of the pull wire 509 coupled to the distal anchor 505.

The tensioner 513 can include various mechanisms for applying and adjusting tension in the pullwire 509. In some implementations, the tensioner 513 can include a rotatable knob coupled to a spool around which the pullwire 509 is wound. Additionally, or alternatively, the tensioner 513 can include linear sliding mechanisms, lever arrangements, or motorized components for tension control. The tensioner 513 can optionally include locking mechanisms to maintain selected tension levels.

As shown in FIG. 5A, when the tensioner 513 applies tension to the pull wire 509, the resilient member 503 is maintained in a compressed state between the distal anchor 505 and proximal anchor 507. The resilient member 503 can be configured with appropriate material properties and geometric parameters to maintain consistent performance characteristics while under compression. In some implementations, the compressed state corresponds to the unextended configuration of the distal end portion 107. When the tensioner 513 reduces or releases tension on the pull wire 509, as shown in FIG. 5B, the resilient member 503 can expand toward its relaxed state, thereby moving the distal anchor 505 distally relative to the proximal anchor 507 and extending the distal end portion 107.

The extension of the distal end portion 107 can be controlled by modulating the tension applied to the pullwire 509 by the tensioner 513. In some implementations, the resilient member 503 can be configured to provide a predetermined extension distance when fully relaxed, such as between about 10 millimeters and about 50 millimeters. Surface treatments or coatings can be applied to the components to reduce friction and wear during repeated extension cycles. Additionally, or alternatively, the tensioner 513 can maintain intermediate levels of tension to achieve partial extension of the distal end portion 107 between the fully compressed and fully extended states, providing precise control over the position of the distal end portion 107 throughout a procedure.

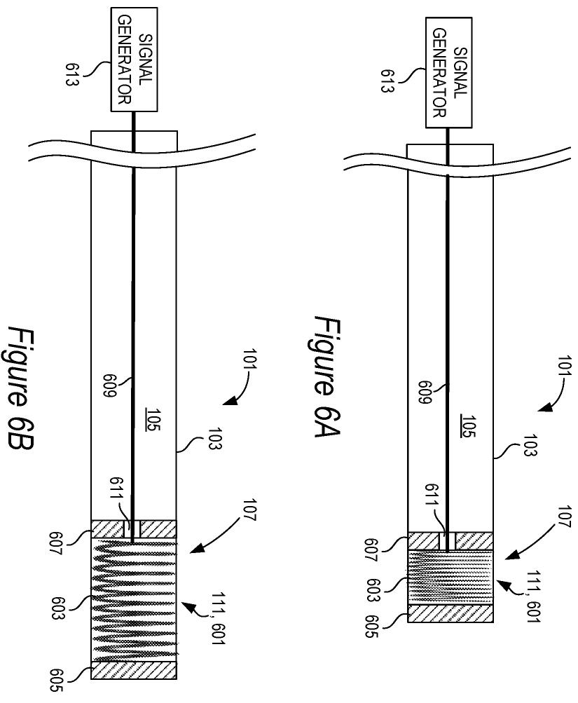

FIGS. 6A and 6B illustrate side cross-sectional views of an implementation of the treatment device 101 having an actuation element 111 configured as a shape-memory assembly 601. FIG. 6A shows the distal end portion 107 in an unextended configuration, while FIG. 6B shows the distal end portion 107 in an extended configuration achieved through thermal activation of a shape-memory element 603.

The shape-memory assembly 601 includes the shape-memory element 603 disposed between a distal anchor 605 and a proximal anchor 607. In some implementations, the shape-memory element 603 can be formed from a shape-memory alloy such as nitinol and configured as a spring or other compressible structure having a predetermined expanded shape. The shape-memory element 603 can be processed to have a transition temperature above normal body temperature but below a temperature that could cause tissue damage, such as between about 42 °C and about 50 °C. Additionally or alternatively, the shape-memory element 603 can be configured with various geometric parameters, such as wire diameter, coil diameter, and number of turns, selected to achieve desired extension characteristics upon activation. The shape-memory element 603 can include or be covered with a biocompatible stretchable polymer material, a jacket, sheath, coating, etc.

A conductor 609 extends through the lumen 105 and is electrically coupled to the shape-memory element 603. The conductor 609 can be formed from materials having suitable electrical conductivity and mechanical properties, such as copper, silver, or other conductive metals, optionally with insulative coatings. The conductor 609 passes through an aperture 611 in the proximal anchor 607 and extends proximally through the tubular body 103 to a signal generator 613 disposed at or near the proximal end of the treatment device 101. In some examples, the conductor 609 can connect to any section of 603 at the distal end. Additionally, conductor 609 can either pass through the lumen or remain outside the lumen (but within the outer jacket) before connecting to the proximal end of the device.

The signal generator 613 can be configured to provide controlled electrical current through the conductor 609 to the shape-memory element 603. In some implementations, the signal generator 613 can include adjustable current output settings to control the rate and extent of heating. Additionally, or alternatively, the signal generator 613 can include temperature monitoring capabilities to maintain the shape-memory element 603 within desired temperature ranges during activation.

As shown in FIG. 6A, the shape-memory element 603 is initially in a compressed state between the distal anchor 605 and proximal anchor 607. When the signal generator 613 supplies current through the conductor 609, resistive heating of the shape-memory element 603 causes its temperature to rise above its transition temperature. As shown in FIG. 6B, this heating activates the shape-memory effect, causing the shape-memory element 603 to transform toward its predetermined expanded shape, thereby moving the distal anchor 605 distally relative to the proximal anchor 607 and extending the distal end portion 107. In various examples, the current can be a simple low-voltage DC current or, more commonly, a pulsed DC current with pulse-width modulation (PWM) module for better control when activating nitinol coils or other shape memory elements.

The extension of the distal end portion 107 can be controlled by modulating the current supplied by the signal generator 613. In some implementations, the shape-memory element 603 can be configured to provide a predetermined extension distance when fully activated, such as between about 10 millimeters and about 50 millimeters. Temperature sensors or other monitoring elements can optionally be incorporated to provide feedback for precise control of the activation process. Additionally or alternatively, the signal generator 613 can provide varying current levels to achieve partial activation and intermediate extension states between the fully compressed and fully extended configurations.

As noted above, the treatment devices described herein can be used to remote clot material from a blood vessel or other bodily lumen. In some implementations, a method of removing a clot from a blood vessel can include navigating the treatment device to a treatment site containing the clot. Initially, the distal end portion can be maintained in an unextended configuration as the treatment device is advanced through the vasculature, which can help facilitate tracking through tortuous anatomy. Upon reaching the treatment site, the distal end portion can be positioned proximal to the clot, and vacuum can be initiated through the lumen to begin engaging the proximal face of the clot.

Once initial engagement with the clot is achieved, the actuation element can be operated to extend the distal end portion distally. In some implementations, this extension can occur during a temporary pause in vacuum application to reduce forces that might otherwise resist the extension. The extension of the distal end portion can allow it to advance further over the clot, effectively increasing the surface area of engagement between the treatment device and the clot. Additionally or alternatively, the extension can be performed incrementally through multiple smaller adjustments coordinated with pulsed vacuum application, progressively engaging more of the clot with each extension.

After achieving desired engagement with the clot, either through partial or complete ingestion into the lumen or through secure contact with the distal end portion, the treatment device can be withdrawn to remove the clot from the blood vessel. In some implementations, vacuum can be maintained during withdrawal to help retain the clot within or against the treatment device. Additionally or alternatively, the distal end portion can be maintained in its extended configuration during withdrawal to help prevent loss of the clot. The increased coverage of the clot provided by the extended distal end portion can help prevent fragmentation or distal embolization during the withdrawal process.

### EXAMPLES

The subject technology is illustrated, for example, according to various aspects described below, including with reference to FIGS. 1–6B. Various examples of aspects of the subject technology are described as numbered examples (1, 2, 3, etc.) for convenience. These are provided as examples and do not limit the subject technology.

Example 1: An aspiration system comprising: a catheter having a proximal end, a distal end, and a lumen extending therebetween; a vacuum source configured to provide vacuum to the lumen; and an actuation mechanism coupled to the distal end of the catheter and configured to selectively extend the distal end in a distal direction by a controlled distance, wherein extension of the distal end facilitates engagement and retrieval of a clot.

Example 2: The aspiration system of Example 1, wherein the actuation mechanism comprises a tension wire extending along the catheter and coupled to the distal end, wherein reducing tension in the tension wire causes the distal end to extend distally.

Example 3: The aspiration system of Example 1, wherein the actuation mechanism comprises a shape memory element disposed at the distal end and configured to extend the distal end distally when heated above a transition temperature.

Example 4: The aspiration system of any one of Examples 1-3, comprising a single lumen extending from the proximal end to the distal end.

Example 5: The aspiration system of any one of Examples 1-3, wherein the catheter comprises: an inner lumen extending from the proximal end to the distal end; and an outer lumen disposed around the inner lumen at the distal end and configured to extend distally relative to the inner lumen.

Example 6: The aspiration system of any one of Examples 1-5, wherein: the vacuum source is configured to provide pulsed vacuum having on and off states; and the actuation mechanism is configured to extend the distal end during vacuum off states.

Example 7: The aspiration system of any one of Examples 1-6, wherein the actuation mechanism is configured to extend the distal end by a distance of between about 10 millimeters and about 50 millimeters.

Example 8: The aspiration system of any one of Examples 1-7, further comprising a flexible polymer jacket disposed at the distal end and configured to stretch during extension of the distal end.

Example 9: An aspiration catheter comprising: an elongate body having a proximal end, a distal end, and a lumen extending therebetween; and an actuation element coupled to the distal end and configured to extend the distal end in a distal direction by a controlled distance in response to actuation; wherein extension of the distal end facilitates engagement with a clot during aspiration through the lumen.

Example 10: The aspiration catheter of Example 9, wherein the actuation element comprises a tension wire extending along the elongate body and coupled to the distal end, wherein reducing tension in the tension wire causes the distal end to extend distally.

Example 11: The aspiration catheter of Example 9, wherein the actuation element comprises a shape memory element configured to extend the distal end distally when heated by an electrical current.

Example 12: The aspiration catheter of any one of Examples 9-11, comprising a single lumen extending from the proximal end to the distal end.

Example 13: The aspiration catheter of any one of Examples 9-11, comprising: an inner lumen extending from the proximal end to the distal end; and an outer lumen disposed around the inner lumen at the distal end and configured to extend distally relative to the inner lumen.

Example 14: The aspiration catheter of any one of Examples 9-13, wherein the actuation element is configured to extend the distal end by a distance of between about 10 millimeters and about 50 millimeters.

Example 15: The aspiration catheter of any one of Examples 9-14, further comprising a stretchable polymer jacket covering the distal end.

Example 16: A method of removing a clot from a blood vessel comprising: advancing a catheter to a target site containing a clot, the catheter having a lumen extending to a distal end; applying vacuum through the lumen; actuating the distal end to extend in a distal direction by a controlled distance; engaging the clot; and withdrawing the catheter to remove the engaged clot from the blood vessel.

Example 17: The method of Example 16, wherein actuating the distal end comprises reducing tension in a tension wire coupled to the distal end.

Example 18: The method of Example 16, wherein actuating the distal end comprises applying electrical current to a shape memory element to heat the shape memory element.

Example 19: The method of any one of Examples 16-18, wherein the lumen is a single lumen extending from the proximal end to the distal end.

Example 20: The method of any one of Examples 16-18, wherein: the catheter comprises an inner lumen and an outer lumen disposed around the inner lumen at the distal end; and actuating the distal end comprises extending the outer lumen distally relative to the inner lumen.

Example 21: The method of any one of Examples 16-20, wherein: applying vacuum comprises providing pulsed vacuum having on and off states; and actuating the distal end occurs during vacuum off states.

Example 22: The method of any one of Examples 16-21, wherein extending the distal end increases coverage of the clot by the distal end.

Example 23: The method of any one of Examples 16-22, wherein actuating the distal end comprises extending the distal end by a distance of between about 10 millimeters and about 50 millimeters.

Example 24: The method of any one of Examples 16-23, wherein the distal end comprises a flexible polymer jacket that stretches during extension of the distal end.

### CONCLUSION

Although many of the embodiments are described above with respect to systems, devices, and methods for treating vessel occlusions in the brain, the technology is applicable to other applications and/or other approaches, such as vessel occlusions elsewhere in the body. As noted herein, in some implementations the treatment devices and systems disclosed herein can be used for delivery of fluid instead of or in addition to aspiration. This can include, for instance, delivery of fluid containing medicament (e.g., any substance used for medical treatment, diagnosis, disease prevention, and/or health promotion). Additionally, catheters as described herein can have applications for access purposes only. For instance, an extendable distal section can be manipulated independently of the proximal section, allowing for direct navigation to a lesion without the need for an additional guidewire. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1–6B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term "comprising" is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An aspiration system comprising:
a catheter having a proximal end, a distal end, and a lumen extending therebetween;
a vacuum source configured to provide vacuum to the lumen; and
an actuation mechanism coupled to the distal end of the catheter and configured to selectively extend the distal end in a distal direction by a controlled distance,
wherein extension of the distal end facilitates engagement and retrieval of a clot.

2. The aspiration system of claim 1, wherein the actuation mechanism comprises a tension wire extending along the catheter and coupled to the distal end, wherein reducing tension in the tension wire causes the distal end to extend distally.

3. The aspiration system of claim 1, wherein the actuation mechanism comprises a shape memory element disposed at the distal end and configured to extend the distal end distally when heated above a transition temperature.

4. The aspiration system of claim 1, comprising a single lumen extending from the proximal end to the distal end.

5. The aspiration system of claim 1, wherein the catheter comprises:
an inner lumen extending from the proximal end to the distal end; and
an outer lumen disposed around the inner lumen at the distal end and configured to extend distally relative to the inner lumen.

6. The aspiration system of claim 1, wherein:
the vacuum source is configured to provide pulsed vacuum having on and off states; and
the actuation mechanism is configured to extend the distal end during vacuum off states.

7. The aspiration system of claim 1, wherein the actuation mechanism is configured to extend the distal end by a distance of between about 10 millimeters and about 50 millimeters.

8. The aspiration system of claim 1, further comprising a flexible polymer jacket disposed at the distal end and configured to stretch during extension of the distal end.

9. An aspiration catheter comprising:
an elongate body having a proximal end, a distal end, and a lumen extending therebetween; and
an actuation element coupled to the distal end and configured to extend the distal end in a distal direction by a controlled distance in response to actuation;
wherein extension of the distal end facilitates engagement with a clot during aspiration through the lumen.

10. The aspiration catheter of claim 9, wherein the actuation element comprises a tension wire extending along the elongate body and coupled to the distal end, wherein reducing tension in the tension wire causes the distal end to extend distally.

11. The aspiration catheter of claim 9, wherein the actuation element comprises a shape memory element configured to extend the distal end distally when heated by an electrical current.

12. The aspiration catheter of claim 9, comprising a single lumen extending from the proximal end to the distal end.

13. The aspiration catheter of claim 9, comprising:
an inner lumen extending from the proximal end to the distal end; and
an outer lumen disposed around the inner lumen at the distal end and configured to extend distally relative to the inner lumen.

14. The aspiration catheter of claim 9, wherein the actuation element is configured to extend the distal end by a distance of between about 10 millimeters and about 50 millimeters.

15. The aspiration catheter of claim 9, further comprising a stretchable polymer jacket covering the distal end.

16. A method of removing a clot from a blood vessel comprising:
advancing a catheter to a target site containing a clot, the catheter having a lumen extending to a distal end;
applying vacuum through the lumen;
actuating the distal end to extend in a distal direction by a controlled distance;
engaging the clot; and
withdrawing the catheter to remove the engaged clot from the blood vessel.

17. The method of claim 16, wherein actuating the distal end comprises reducing tension in a tension wire coupled to the distal end.

18. The method of claim 16, wherein actuating the distal end comprises applying electrical current to a shape memory element to heat the shape memory element.

19. The method of claim 16, wherein:
the catheter comprises an inner lumen and an outer lumen disposed around the inner lumen at the distal end; and
actuating the distal end comprises extending the outer lumen distally relative to the inner lumen.

20. The method of claim 16, wherein:
applying vacuum comprises providing pulsed vacuum having on and off states; and
actuating the distal end occurs during vacuum off states.
